(19) United States
(12) Patent Application Publication
(10) Pub. No.: US 2018/0280001 A1
(43) Pub. Date: Oct. 4, 2018

(54) MEDICAL IMAGE DIAGNOSIS APPARATUS

(71) Applicant: Canon Medical Systems Corporation, Otawara-shi (JP)

(72) Inventors: Nobuyuki Iwama, Nasushiobara (JP); Shuta Fujiwara, Nasushiobara (JP); Isao Uchiumi, Nasushiobara (JP); Hiroyuki Shibanuma, Yaita (JP); Wataru Kameishi, Nasushiobara (JP)

(73) Assignee: Canon Medical Systems Corporation, Otawara-shi (JP)

(21) Appl. No.: 15/941,045

(22) Filed: Mar. 30, 2018

(30) Foreign Application Priority Data
Mar. 30, 2017 (JP) 2017-068843

(51) Int. Cl. A61B 8/00; H02M 1/42; A61B 6/00

(52) U.S. Cl. CPC A61B 8/56; H02M 2001/0012; A61B 6/56; H02M 1/42

(57) ABSTRACT

A medical image diagnosis apparatus includes a power factor corrector, a DC/DC converter, and a battery device. The power factor corrector is supplied with AC power and generates DC power to drive individual parts of the apparatus. The DC/DC converter converts the voltage of the DC power generated by the power factor corrector to a voltage desired for driving the individual parts. The battery device includes a battery that stores DC power, a discharging circuit that is connected to the downstream side of the power factor corrector and supplies the DC power from the battery to the individual parts, and a charging circuit that is connected to the upstream side of the power factor corrector, and is supplied with AC power and supplies DC power to the battery. The battery device supplies the DC power to the individual parts when the power factor corrector cannot supply the DC power.

### CROSS-REFERENCE TO RELATED APPLICATION

This application is based upon and claims the benefit of priority from Japanese Patent Application No. 2017-068843, filed May 30, 2017; the entire contents of which are incorporated herein by reference.

### FIELD

Embodiments described herein relate generally to a medical image diagnosis apparatus.

### BACKGROUND

In recent years, a medical image diagnosis apparatus has been used to collect information on the inside of a subject and image the inside of the subject based on the information to generate a medical image. Examples of the medical image diagnosis apparatus include an ultrasound diagnosis apparatus, an X-ray computed tomography (CT) system, magnetic resonance imaging (MRI) equipment, and the like.

Generally, the medical image diagnosis apparatus is installed in, for example, an examination room in a medical institution, and used in a stationary state. Accordingly, in order to conduct an examination or the like using the medical image diagnosis apparatus, a patient to be examined has to go to the place where the medical image diagnosis apparatus is installed.

Besides, if installed in a stationary state, the medical image diagnosis apparatus is supplied with alternating current (AC) power through its plug connected to a socket. However, there may be cases where the power supply is stopped in an unexpected event such as a power failure. Therefore, for example, some medical image diagnosis apparatuses are provided with a battery device on both the inside and outside thereof.

On the other hand, there has been proposed a medical image diagnosis apparatus configured to be movable so that it can also be used for a patient who has been unable to go to the place where the apparatus is installed. If the medical image diagnosis apparatus is portable, the apparatus is not always supplied with AC power from a socket as in the case of the stationary one. In other words, the plug needs to be removed from the socket to move the medical image diagnosis apparatus, and the apparatus has to rely on power supply from the battery device until the plug is inserted into a socket again.

If the battery drains too fast while the medical image diagnosis apparatus is being suppled with power from the battery device, the apparatus cannot be used stably. In particular, when the battery device is for emergency use, charging of the battery device is often not taken into consideration. In addition, even when the medical image diagnosis apparatus is configured to be movable, if the battery cannot be fully charged, the apparatus cannot sufficiently perform the role as a portable medical image diagnosis apparatus.

### BRIEF DESCRIPTION OF THE DRAWINGS

FIG. 1 is a block diagram illustrating the overall configuration of a medical image diagnosis apparatus according to a first embodiment;

FIG. 2 is a block diagram illustrating the overall configuration of a medical image diagnosis apparatus according to a second embodiment; and

FIG. 3 is a block diagram illustrating the overall configuration of a medical image diagnosis apparatus according to a third embodiment.

### DETAILED DESCRIPTION

In general, according to one embodiment, a medical image diagnosis apparatus includes a power factor corrector, a DC/DC converter, and a battery device. The power factor corrector is supplied with AC power and generates DC power to drive individual parts of the apparatus. The DC/DC converter converts the voltage of the DC power generated by the power factor corrector to a voltage desired for driving the individual parts. The battery device includes a battery that stores DC power, a discharging circuit that is connected to the downstream side of the power factor corrector and supplies the DC power from the battery to the individual parts, and a charging circuit that is connected to the upstream side of the power factor corrector, and is supplied with AC power and supplies DC power to the battery. The battery device supplies the DC power to the individual parts when the power factor corrector cannot supply the DC power to the individual parts.

Exemplary embodiments are directed in detail with reference to the drawings.

### First Embodiment

### [Configuration of Medical Image Diagnosis Apparatus]

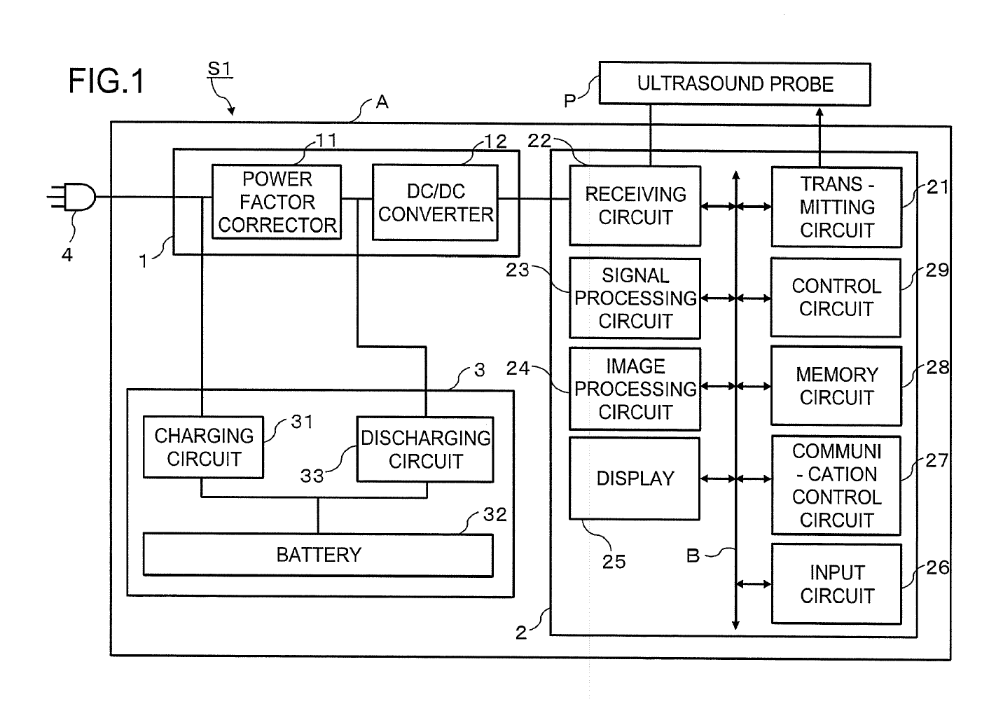

FIG. 1 is a block diagram illustrating the overall configuration of a medical image diagnosis apparatus according to a first embodiment. In the following, an ultrasound image diagnosis apparatus is described as an example of the medical image diagnosis apparatus.

The ultrasound image diagnosis apparatus transmits ultrasound waves toward the inside of a subject from an ultrasound probe having transducers (piezoelectric transducers) at the tip. The ultrasound image diagnosis apparatus receives reflected waves caused by acoustic impedance mismatch inside the subject through the transducers of the ultrasound probe. The ultrasound image diagnosis apparatus generates an ultrasound image based on received signals thus obtained.

An ultrasound image diagnosis apparatus S1 of the first embodiment includes an ultrasound probe P configured to transmit and receive ultrasound waves (transmit/receive waves) to and from a subject, and a main body A. The ultrasound probe P is detachably connected to the main body A.

The ultrasound probe P transmits ultrasound waves to the inside of the subject from each of ultrasound transducers to scan a scan area, and receives reflected waves from the subject as echo signals. Examples of the ultrasound probe P include sector scan probes, linear scan probes, convex scan probes, and the like, one of which is arbitrarily selected according to the site to be diagnosed.

The ultrasound transducers need not necessarily be one-dimensionally arrayed. With the ultrasound transducers arranged two-dimensionally, volume data can be acquired in real time. In the case of obtaining a three-dimensional stereoscopic image, a three-dimensional scanning probe is used as the ultrasound probe P. Examples of the three-dimensional scanning probe include two-dimensional array probes and mechanical four-dimensional probes.

The main body A includes a power supply 1, an operation unit 2, a battery device 3, and a plug 4. When the plug 4 is inserted into a socket, the ultrasound image diagnosis apparatus S1 is supplied with AC power and thereby driven.

The power supply 1 includes a power factor corrector 11 and a direct current to direct current (DC/DC) converter 12. The power factor corrector 11 is provided to suppress harmonic current generated in an input current. When supplied with AC power through the plug 4, the power factor corrector 11 generates DC power to drive each part of the ultrasound image diagnosis apparatus S1. That is, the power factor corrector serves as an AC/DC converter. The DC/DC converter 12 converts the DC power generated by the power factor corrector 11 to a voltage suitable for driving the operation unit 2 of the ultrasound image diagnosis apparatus S1.

The operation unit 2 is driven when supplied with power from the power supply 1. In FIG. 1, a single solid line indicates the supply of power from the power supply 1 to the operation unit 2. However, as described above, the DC/DC converter 12 has a function of converting the power into a driving voltage suitable for each part of the operation unit 2. Accordingly, the line indicating the supply of power from the power supply 1 to the operation unit 2 represents each line that connects the DC/DC converter 12 and each part of the operation unit 2 such as, for example, a transmitting circuit 21, a receiving circuit 22, or the like.

The operation unit 2 includes the transmitting circuit 21, the receiving circuit 22, a signal processing circuit 23, an image processing circuit 24, a display 25, and an input circuit 26. The transmitting circuit 21 transmits a drive signal to the ultrasound probe P. The receiving circuit 22 receives an echo signal, i.e., a reflected signal, from the ultrasound probe P. The signal processing circuit 23 processes the echo signal. The image processing circuit 24 generates an ultrasound image. The display 25 displays various images including the ultrasound image. The input circuit 26 receives a signal input by being operated by an operator such as an examiner. The operation unit 2 further includes a communication control circuit 27 that controls the exchange of signals with other devices (not illustrated), a memory circuit 28, and a control circuit 29 that controls each part. These circuits are connected to a bus B and can exchange various signals. Detailed functions of the circuits are described below.

Under the control of the control circuit 29, the transmitting circuit 21 generates a drive signal for causing the ultrasound probe P to generate ultrasound waves, i.e., an electric pulse signal to be applied to each of the piezoelectric transducers (hereinafter referred to as “drive pulse”), and transmits the driving pulse to the ultrasound probe P. The transmitting circuit 21 includes circuits such as a reference pulse generating circuit, a delay control circuit, a drive pulse generating circuit, and the like (not illustrated), and each circuit performs the above functions.

The receiving circuit 22 receives a reflected signal from the ultrasound probe P, i.e., an echo signal, and performs phasing addition on the received signal. The receiving circuit 22 outputs the signal obtained by the phasing addition to the signal processing circuit 23.

The signal processing circuit 23 generates various data by using the received signal from the ultrasound probe P fed by the receiving circuit 22, and outputs the data to the image processing circuit 24 and the control circuit 29. The signal processing circuit 23 includes, for example, a B mode processing circuit (or Bc mode processing circuit), a Doppler mode processing circuit, a color Doppler mode processing circuit, and the like (not illustrated). The B mode processing circuit visualizes the amplitude information of the received signal and generates data based on a B mode signal. The Doppler mode processing circuit extracts the Doppler shift frequency component from the received signal, and performs fast Fourier transform (FFT) and the like to generate Doppler signal data of blood flow information. The color Doppler mode processing circuit visualizes the blood flow information based on the received signal, and generates data based on a color Doppler mode signal.

The image processing circuit 24 generates two-dimensional or three-dimensional ultrasound images of the scan area based on the data received from the signal processing circuit 23. For example, the image processing circuit 24 generates volume data on the scan area based on the data. Then, from the volume data generated, the image processing circuit 24 generates two-dimensional ultrasound image data by multi-planar reconstruction (MPR) or three-dimensional ultrasound image data by volume rendering. The image processing circuit 24 outputs the two-dimensional or three-dimensional ultrasound image to the display 25. Examples of the ultrasound image include a B mode image, a Doppler mode image, a color Doppler mode image, an M mode image, and the like.

The display 25 displays various images such as the ultrasound image generated by the image processing circuit 24 and an operation screen (for example, graphical user interface (GUI) for receiving various instructions from the operator) under the control of the control circuit 29. As the display 25, for example, a liquid crystal display, an organic electroluminescence (EL) display, or the like can be used.

The input circuit 26 receives various instructions to, for example, display an image, switch images, and designate the mode and various settings, provided by the operator. Examples of the input circuit 26 include such input devices as GUT, buttons, a keyboard, a trackball, and a touch panel displayed on the display 25.

In the embodiment, the display 25 and the input circuit 26 are each described as one constituent element of the ultrasound image diagnosis apparatus S1; however, it is not so limited. For example, the display 25 need not necessarily be a component of the ultrasound image diagnosis apparatus S1 and may be separated therefrom. Further, the input circuit can be a touch panel using the separate display.

The communication control circuit 27 has a function of, for example, connecting the ultrasound image diagnosis apparatus S1 to medical image diagnosis apparatuses (modalities), servers, workstations, and the like (not illustrated), which are connected to a communication network (not illustrated). The communication control circuit 27 may use any standard such as digital imaging and communication in medicine (DICOM) for exchanging information and medical images with other equipment via the communication network.

The memory circuit 28 is formed of, for example, a semiconductor or a magnetic disk, and stores programs executed by the control circuit 29, data, and the like.

The control circuit 29 comprehensively controls each part of the ultrasound image diagnosis apparatus S1. The control circuit 29 performs, for example, a process desired by the operator. Further, for example, the control circuit 29 causes the display 25 to display the ultrasound image generated by the image processing circuit 24.

The battery device 3 is connected to the power supply 1. The battery device 3 supplies DC power to each part when the power supply 1 cannot supply DC power thereto. For example, the power supply 1 cannot supply DC power when there is no supply of AC power through the plug 4 due to a power failure or the like, when the plug 4 is removed from the socket to use the ultrasound image diagnosis apparatus S1 in another place, and the like.

The battery device 3 of the embodiment includes therein a charging circuit 31, a battery 32, and a discharging circuit 33. Having been supplied with AC power, the charging circuit 31 supplies DC power to the battery 32. The battery 32 stores electric power. The discharging circuit 33 supplies DC power output from the battery 32 to the operation unit 2.

Note that the circuit configuration of the charging circuit 31 or the discharging circuit 33 is not particularly limited, and a known configuration can be employed. Similarly, any configuration can be arbitrarily used for the battery 32.

The charging circuit 31 is connected to the upstream side of the power factor corrector 11, and directly receives the supply of AC power from the plug 4 without through the power factor corrector 11. Since the battery 32 outputs DC power to the discharging circuit 33, the charging circuit 31 of the embodiment has the function of an AC/DC converter. That is, having been supplied with AC power from the plug 4, the charging circuit 31 converts it into DC power, and outputs the DC power to the battery 32. In this manner, the charging circuit 31 converts AC power into DC power, and outputs it to the battery 32. However, the DC voltage of the battery 32 is lower than the voltage of the AC power. Therefore, the charging circuit 31 has the function of, for example, a step-down AC/DC converter.

The battery 32 is supplied with DC power from the charging circuit 31 and stores it. The battery 32 supplies the operation unit 2 of the ultrasound image diagnosis apparatus S1 with the DC power stored. The DC power is supplied via the discharging circuit 33.

The discharging circuit 33 is connected to the downstream side of the power factor corrector 11, and supplies the DC power received from the battery 32 to the operation unit 2 via the DC/DC converter 12. As described above, the discharging circuit 33 is supplied with DC power from the battery 32 and outputs also DC power. Therefore, the discharging circuit 33 has the function of a DC/DC converter. Further, since the voltage of the DC power supplied from the battery 32 is lower than the voltage between the power factor corrector 11 and the DC/DC converter 12, the discharging circuit 33 has the function of, for example, a step-up DC/DC converter.

### [Operation]

As described above, the battery device 3 is connected to the power supply 1. Accordingly, DC power is supplied to the operation unit 2 of the ultrasound image diagnosis apparatus S1 in the following manner.

First, when the plug 4 is connected to a socket and the ultrasound image diagnosis apparatus S1 is supplied with AC power, the AC power is supplied to the power supply 1 through the plug 4. The AC power is fed to the power factor corrector 11, converted to DC power, and input to the DC/DC converter 12. The DC/DC converter 12 adjusts the voltage of the DC power to be suitable for each circuit of the operation unit 2, and supplies the DC power to each part.

As the plug 4 is connected to the socket, the AC power input through the plug 4 is also supplied to the charging circuit 31 of the battery device 3. The charging circuit 31 converts the AC power into DC power and steps down the voltage. The battery 32 stores the DC power.

On the other hand, when the plug 4 is removed from the socket, or even if the plug 4 is connected to a socket, in the case of an emergency such as, for example, a power failure, AC power is not supplied to the ultrasound image diagnosis apparatus S1. In this case, the DC power stored in the battery 32 is supplied to the power supply 1 via the discharging circuit 33.

The discharging circuit 33 boosts the DC power received from the battery 32 and supplies the DC power to the DC/DC converter 12 of the power supply 1. In this manner, even when the ultrasound image diagnosis apparatus S1 is not supplied with AC power, the battery device 3 supplies DC power to the operation unit 2, thereby enabling the use of the ultrasound image diagnosis apparatus S1.

As described above, a medical image diagnosis apparatus, which has a simple structure while equipped with a rechargeable battery device, can be provided at a low cost. Particularly, in this embodiment, the battery device 3 is provided with the charging circuit 31 therein, and power is constantly stored in the battery 32 while the ultrasound image diagnosis apparatus S1 is being supplied with AC power. Therefore, the ultrasound image diagnosis apparatus S1 can be used in an emergency, and further can be moved for use elsewhere by removing the plug 4 from a socket.

In addition, by using such a configuration, even when the ultrasound image diagnosis apparatus S1 is off, the ultrasound image diagnosis apparatus S1 is supplied with AC power if the plug 4 is inserted in a socket. Therefore, the charging circuit 31 can be charged even during the time when the ultrasound image diagnosis apparatus S1 is not used.

Further, in this embodiment, at least the discharging circuit 33 of the battery device 3 is connected between the power factor corrector 11 and the DC/DC converter 12 of the power supply 1. When power is directly supplied from the battery device 3 to the operation unit 2, for example, it is necessary to increase the charge/discharge current capacity. This inevitably leads to an increase in the size of the battery device 3. Therefore, instead of directly connecting the battery device 3 to the operation unit 2, power is supplied from the battery device 3 to the operation unit 2 via the power supply 1. Thereby, the size of the battery device 3 can be reduced. In addition, since less amount of current is required, the loss is small. As a result, the charge/discharge efficiency of the battery device 3 can be improved.

### Second Embodiment

Next, a second embodiment is described. In the second embodiment, the same parts as described in the first embodiment are denoted by like reference numerals, and the same description is not repeated.

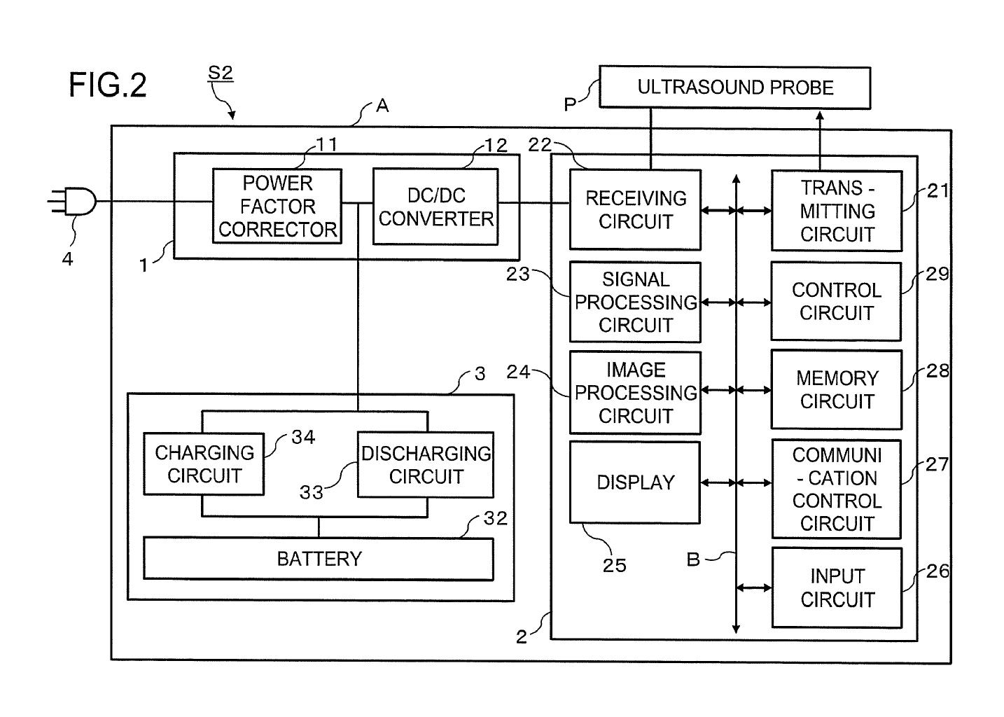

FIG. 2 is a block diagram illustrating the overall configuration of an ultrasound image diagnosis apparatus S2 as the medical image diagnosis apparatus of the second embodiment. The second embodiment is different from the first embodiment in the power feed path to the charging circuit in the battery device 3.

In the first embodiment, the charging circuit 31 is connected to the upstream side of the power factor corrector 11 and is supplied with AC power. In the second embodiment, a charging circuit 34 is connected to the downstream side of the power factor corrector 11 and is supplied with DC power from the power factor corrector 11.

The charging circuit 34 of the embodiment has the function of a DC/DC converter. This is because the power factor corrector 11 converts AC power into DC power, and the charging circuit 34 is supplied with the DC power from the power factor corrector 11. Besides, the voltage of power stored in the battery 32 is lower than the input voltage from the power factor corrector 11. Therefore, the charging circuit 34 steps down the voltage of the DC power received from the power factor corrector 11, and supplies it to the battery 32.

Further, as illustrated in FIG. 2, the power supply 1 and the battery device 3 are connected by a single power line. Therefore, when the ultrasound image diagnosis apparatus S2 is supplied with AC power through the plug 4, the charging circuit 34 is supplied with DC power from the power factor corrector 11, and stores the power in the battery 32. On the other hand, in the case of an emergency such as a power failure or when the plug 4 is removed from a socket to use the ultrasound image diagnosis apparatus S2 in another place, the discharging circuit 33 supplies DC power from the battery 32 to the operation unit 2 via the DC/DC converter 12.

As described above, a medical image diagnosis apparatus, which has a simple structure while equipped with a rechargeable battery device, can be provided at a low cost. In this embodiment also, the battery device 3 is provided with the charging circuit 31 therein, and power is constantly stored in the battery 32 while the ultrasound image diagnosis apparatus S2 is being supplied with AC power. Therefore, the ultrasound image diagnosis apparatus S2 can be used in an emergency, and further can be moved for use elsewhere by removing the plug 4 from a socket.

Besides, the charging circuit 34 of the embodiment uses a power supply line connected to the downstream side of the power factor corrector 11. Since the discharging circuit 33 also uses the power supply line, the connection between the power supply 1 and the battery device 3 is simplified. Accordingly, the routing of the wiring is simplified, which contributes to the downsizing and cost reduction of the apparatus.

### Third Embodiment

Next, a third embodiment is described. In the third embodiment, the same parts as described in the first or second embodiment are denoted by like reference numerals, and the same description is not repeated.

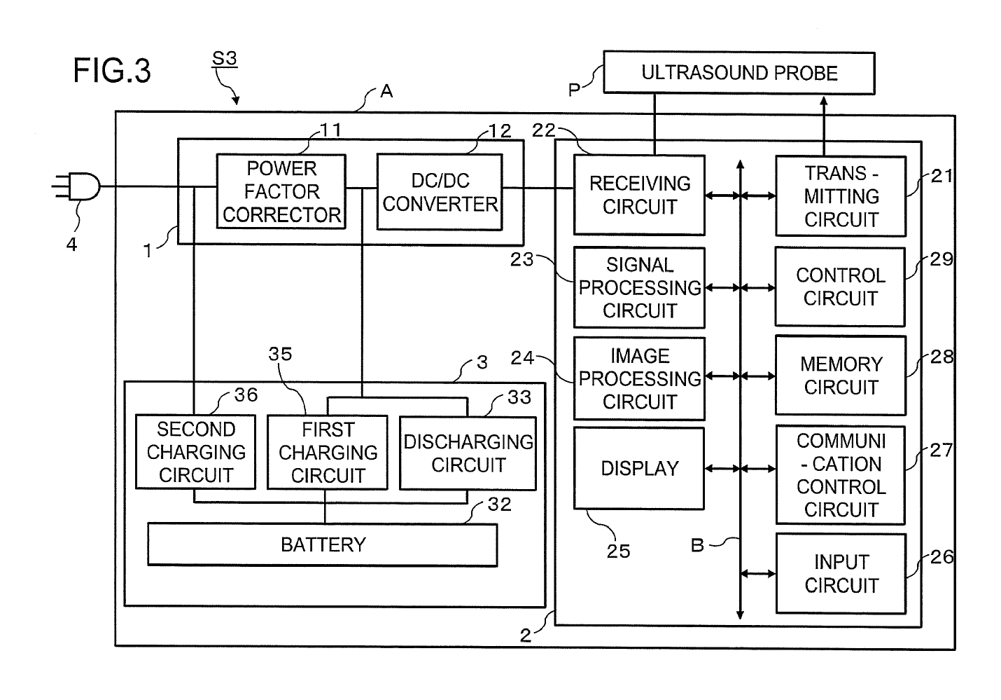

FIG. 3 is a block diagram illustrating the overall configuration of an ultrasound image diagnosis apparatus S3 as the medical image diagnosis apparatus of the third embodiment. The third embodiment is different from the first embodiment or the second embodiment in the power feed path to the charging circuit in the battery device 3.

In the third embodiment, the battery device 3 includes two charging circuits, i.e., a first charging circuit 35 and a second charging circuit 36. The first charging circuit 35 is connected to the downstream side of the power factor corrector 11 as with the discharging circuit 33, and is supplied with DC power from the power factor corrector 11. The second charging circuit 36 is connected to the upstream side of the power factor corrector 11, and is directly supplied with AC power through the plug 4 instead of receiving the supply of DC power from the power factor corrector 11.

The first charging circuit 35 is supplied with DC power from the power factor corrector 11, and therefore has the function of a DC/DC converter. On the other hand, the second charging circuit 36 directly receives the supply of AC power without through the power factor corrector 11. Thus, the second charging circuit 36 has the function of an AC/DC converter.

As described above, the battery device 3 of the third embodiment includes the first charging circuit 35 and the second charging circuit 36 as two systems capable of charging the battery 32. Therefore, when the ultrasound image diagnosis apparatus S3 is connected to the socket through the plug 4, the battery 32 can be charged via the first charging circuit 35 and the second charging circuit 36.

As described above, a medical image diagnosis apparatus, which has a simple structure while equipped with a rechargeable battery device, can be provided at a low cost. In this embodiment also, the battery device 3 is provided with the first charging circuit 35 and the second charging circuit 36 therein, and power is constantly stored in the battery 32 while the ultrasound image diagnosis apparatus S3 is being supplied with AC power. Therefore, the ultrasound image diagnosis apparatus S3 can be used in an emergency, and further can be moved for use elsewhere by removing the plug 4 from a socket.

Besides, in this embodiment, the battery device 3 is provided with two charging circuits, i.e., the first charging circuit 35 and the second charging circuit 36. Thus, the battery 32 can be charged using the two charging systems. As a result, the charging of the battery 32 can be completed in less time as compared to the case of only one charging system. Moreover, because of the separate two charging systems, the heat generated during charging can be dispersed.

In addition, by using such a configuration, even when the ultrasound image diagnosis apparatus S3 is off, the ultrasound image diagnosis apparatus S3 is supplied with AC power if the plug 4 is inserted in a socket. Therefore, at least the second charging circuit 36 can be charged even during the time when the ultrasound image diagnosis apparatus S3 is not used.

In the battery devices of the first to third embodiments described above, the charging circuit and the discharging circuit are separately provided. However, the battery device may be provided with a so-called bidirectional converter having both the functions of a charging circuit and a discharging circuit.

The output voltage of the discharging circuit to supply power from the battery device may be set slightly lower than the output voltage of the power factor corrector which is supplied with AC power and outputs it to the DC/DC converter. With this setting, for example, when the supply of AC power is stopped and each part of the apparatus is supplied with power from the battery device, power is automatically supplied from the battery device to each part of the apparatus without a special configuration such as a changeover switch or the like for switching the power supply route. This eliminates the need of extra components and contributes to cost reduction and miniaturization.

While certain embodiments have been described, these embodiments have been presented by way of example only, and are not intended to limit the scope of the inventions. Indeed, the novel embodiments described herein may be embodied in a variety of other forms; further, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A medical image diagnosis apparatus, comprising:
a power factor corrector configured to be supplied with alternating current (AC) power and generate direct current (DC) power to drive individual parts of the apparatus;
a DC/DC converter configured to convert voltage of the DC power generated by the power factor corrector to a voltage desired for driving the individual parts; and
a battery device configured to supply DC power to the individual parts when the power factor corrector cannot supply the DC power to the individual parts, wherein the battery device includes
a battery configured to store the DC power,
a discharging circuit connected to a downstream side of the power factor corrector, and configured to supply the DC power from the battery to the individual parts, and
a charging circuit connected to an upstream side of the power factor corrector, and configured to be supplied with AC power and supply the DC power to the battery.

2. A medical image diagnosis apparatus, comprising:
a power factor corrector configured to be supplied with AC power and generate DC power to drive individual parts of the apparatus;
a DC/DC converter configured to convert voltage of the DC power generated by the power factor corrector to a voltage desired for driving the individual parts; and
a battery device configured to supply the DC power to the individual parts when the power factor corrector cannot supply the DC power to the individual parts, wherein the battery device includes
a battery configured to store the DC power,
a discharging circuit connected to a downstream side of the power factor corrector, and configured to supply the DC power from the battery to the individual parts, and
a charging circuit connected to the downstream side of the power factor corrector, and configured to supply the DC power generated by the power factor corrector to the battery.

3. The medical image diagnosis apparatus of claim 2, wherein the battery device further includes a second charging circuit connected to an upstream side of the power factor corrector, and configured to be supplied with AC power and supply the DC power to the battery in addition to the charging circuit connected to the downstream side of the power factor corrector as a first charging circuit.

4. The medical image diagnosis apparatus of claim 2, wherein the charging circuit is further configured to serve as a DC/DC converter that steps down voltage of the DC power received from the power factor corrector and outputs the DC power to the battery.

5. The medical image diagnosis apparatus of claim 3, wherein the first charging circuit is further configured to serve as a DC/DC converter that steps down voltage of the DC power received from the power factor corrector and outputs the DC power to the battery.

6. The medical image diagnosis apparatus of claim 1, wherein the charging circuit is further configured to serve as an AC/DC converter that converts the AC power to DC power and supplies the DC power to the battery.

7. The medical image diagnosis apparatus of claim 3, wherein the second charging circuit is further configured to serve as an AC/DC converter that converts the AC power to DC power and supplies the DC power to the battery.

8. The medical image diagnosis apparatus of claim 1, wherein the discharging circuit is further configured to serve as a step-up DC/DC converter that boosts voltage of the DC power received from the battery and outputs the power.

9. The medical image diagnosis apparatus of claim 2, wherein the discharging circuit is further configured to serve as a step-up DC/DC converter that boosts voltage of the DC power received from the battery and outputs the power.

10. The medical image diagnosis apparatus of claim 3, wherein the discharging circuit is further configured to serve as a step-up DC/DC converter that boosts voltage of the DC power received from the battery and outputs the power.

11. The medical image diagnosis apparatus of claim 1, wherein output voltage of the discharging circuit is set lower than output voltage of the DC power factor corrector.

12. The medical image diagnosis apparatus of claim 2, wherein output voltage of the discharging circuit is set lower than output voltage of the DC power factor corrector.

13. The medical image diagnosis apparatus of claim 3, wherein output voltage of the discharging circuit is set lower than output voltage of the DC power factor corrector.
